(19) United States
(12) Patent Application Publication
(10) Pub. No.: US 2021/0142480 A1
(43) Pub. Date: May 13, 2021

(54) DATA PROCESSING METHOD AND APPARATUS

(71) Applicant: Canon Medical Systems Corporation, Otawara-shi (JP)

(72) Inventors: Paul THOMSON, Edinburgh (GB); Brian MOHR, Edinburgh (GB)

(73) Assignee: Canon Medical Systems Corporation, Otawara-shi (JP)

(21) Appl. No.: 16/680,700

(22) Filed: Nov. 12, 2019

(51) Int. Cl. G06T 7/11; G06T 7/136; G06T 7/00; G16H 30/40; G16H 50/20

(52) U.S. Cl. CPC G06T 7/11; G06T 7/136; G06T 7/0012; G16H 30/40; G06T 2207/20081; G06T 2207/10081; G06T 2207/30016; G06T 2207/20084; G16H 50/20

(57) ABSTRACT

A data processing apparatus comprises processing circuitry configured to: apply a process to a medical data set to obtain multiply-valued and/or continuously-valued process outputs; determine at least one threshold value dependent on patient-specific clinical information; and apply the determined at least one threshold value to the multiply-valued and/or continuously-valued process outputs to obtain thresholded process outputs.

### FIELD

Embodiments described herein relate generally to a data processing method and apparatus, for example a method and apparatus for segmentation of medical imaging data using a clinically-informed threshold.

### BACKGROUND

It is known to perform a segmentation task using a deep learning algorithm, for example a convolutional neural network. For example, a deep learning algorithm may be used to perform a segmentation of the voxels of a medical image data volume into two or more classes, each representative of a respective tissue type.

Typically, the deep learning algorithm may output probability values. The deep learning algorithm may output a probability volume that comprises, for each voxel in an image volume, a probability that the voxel belongs to one or more classes.

We consider the case of a binary classification task, in which the classification task to be performed is a classification into two classes, for example two tissue types. The deep learning algorithm outputs a probability volume comprising a set of probability values. A threshold is then applied to the probability volume to obtain a binary mask. The binary mask includes the voxels that are classified as being in the first class, and excludes the voxels that are classified as being in the second class.

Thresholding a probability volume is a very common way to get a binary mask from a deep learning algorithm. The probability values in the probability volume are each thresholded to obtain a classification as the first class or the second class of the two classes. For example, a threshold may be set at a probability of 0.5. Voxels having a probability value of greater than 0.5 may be classified as belonging to the first class, and included in the binary mask. All other voxels may be classified as belonging to the second class, and excluded from the binary mask.

Normally, the threshold to be applied to the probability values is selected only once. For example, the threshold may be selected during algorithm development.

One common method of threshold selection is to use the half-way point of probability=0.5 as described above.

Another method of threshold selection is to use a distance from an optimal classifier on an ROC (Receiver Operating Curve). An ROC plots true positive rate against false positive rate at multiple threshold settings as applied to probability values that are output by a binary classification. By changing the threshold value, changes may be made to the true positive rate and/or to the false positive rate. An optimal classifier may be a hypothetical classifier that is always right and is never wrong. An optimal classifier may have no false positives and no false negatives.

Another method of threshold selection is to select the threshold using Youden's index. Youden's index is a function of sensitivity (true positive rate) and specificity (true negative rate).

Another method of threshold selection is to select a threshold based on the relative costs of true and false predictions. The costs may include, for example, financial cost, patient welfare and/or prevalence (where prevalence is the proportion of a population having a medical condition).

Another method of threshold selection is manual selection, for example by a doctor at the time of use. For example, a doctor may choose a preferred combination of sensitivity and specificity.

Another method of threshold selection is to perform automatic threshold selection based on a predicted probability volume.

In some circumstances, the use of a single threshold may not be appropriate to all clinical scenarios in which the task is to be performed. The ideal threshold may vary based on actual model performance, desired performance, clinical use case and/or other factors.

### BRIEF DESCRIPTION OF THE DRAWINGS

Embodiments are now described, by way of non-limiting example, and are illustrated in the following figures, in which:

FIG. 1 is a schematic diagram of an apparatus according to an embodiment;

FIG. 2 is a flow chart illustrating in overview a method of an embodiment; and

FIG. 3 is a flow chart illustrating in overview a training method according to an embodiment.

### DETAILED DESCRIPTION

Certain embodiments provide a data processing apparatus comprising processing circuitry configured to: apply a process to a medical data set to obtain multiply-valued and/or continuously-valued process outputs; determine at least one threshold value dependent on patient-specific clinical information; and apply the determined at least one threshold value to the multiply-valued and/or continuously-valued process outputs to obtain thresholded process outputs.

Certain embodiments provide a method for processing medical data comprising: applying a process to a medical data set to obtain multiply-valued and/or continuously-valued process outputs; determining at least one threshold value dependent on patient-specific clinical information; and applying the determined at least one threshold value to the multiply-valued and/or continuously-valued process outputs to obtain thresholded process outputs.

Certain embodiments provide a training apparatus comprising processing circuitry configured to: obtain training data comprising patient-specific clinical information; and train a machine learning algorithm to predict threshold values for multiply-valued and/or continuously-valued outputs of a process by performing a training process using the training data, the training process comprising training the machine learning algorithm to predict threshold values based on patient-specific clinical information.

A data processing apparatus 10 according to an embodiment is illustrated schematically in FIG. 1. The data processing apparatus 10 comprises a computing apparatus 12, in this case a personal computer (PC) or workstation, which is connected to a scanner 14 via a data store 20.

The data processing apparatus 10 further comprises one or more display screens 16 and an input device or devices 18, such as a computer keyboard, mouse or trackball.

In the present embodiment, the scanner 14 is a CT (computed tomography) scanner which is configured to obtain volumetric CT scans, for example coronary CTA scans. In other embodiments, the scanner 14 may be any scanner that is configured to perform medical imaging. The scanner 14 is configured to generate image data that is representative of at least one anatomical region of a patient or other subject.

The scanner 14 may be configured to obtain two-, three- or four-dimensional image data in any imaging modality. For example, the scanner 14 may comprise a magnetic resonance (MR) scanner, CT (computed tomography) scanner, cone-beam CT scanner, X-ray scanner or ultrasound scanner.

In the present embodiment, image data sets obtained by the scanner 14 are stored in data store 20 and subsequently provided to computing apparatus 12. In an alternative embodiment, image data sets are supplied from a remote data store (not shown). The data store 20 or remote data store may comprise any suitable form of memory storage. In some embodiments, the data processing apparatus 10 is not coupled to any scanner.

Computing apparatus 12 comprises a processing apparatus 22 for processing of data. The processing apparatus comprises a central processing unit (CPU) and Graphical Processing Unit (GPU). The processing apparatus 22 provides a processing resource for automatically or semi-automatically processing image data sets.

The processing apparatus 22 includes probability circuitry 24 configured to process data using a deep learning model to obtain a probability volume; threshold circuitry 26 configured to determine a threshold and to apply the threshold to the probability volume; and rendering circuitry 28 configured to render an image using the thresholded probabilities. In the present embodiment, the processing apparatus 22 further comprises training circuitry 29 for training a threshold function. In other embodiments, the various circuitries may be provided on two or more apparatuses. For example, the training circuitry 29 may form part of a separate apparatus from the probability circuitry 24, threshold circuitry 26 and/or rendering circuitry 28.

In other embodiments, the data to be processed may not comprise imaging data. For example, the data to be processed may comprise any medical data.

The probability circuitry 24 may be configured to perform any process that has a continuously-valued output. In other embodiments, the probability circuitry 24 may be configured to perform any process that has a multiply-valued output, for example an output that comprises a set of monotonic discrete values. In further embodiments, the process may be any process that has a multiply-valued or continuously-valued output. In some embodiments, the multiply-valued or continuously-valued output may not comprise probabilities.

In the present embodiment, the circuitries 24, 26, 28, 29 are each implemented in the CPU and/or GPU by means of a computer program having computer-readable instructions that are executable to perform the method of the embodiment. In other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

The apparatus of FIG. 1 is configured to perform a method of processing medical imaging data as illustrated in FIG. 2. In other embodiments, the apparatus of FIG. 1 may be configured to process any medical data.

At stage 30, the probability circuitry 24 obtains a set of volumetric medical imaging data. The set of medical imaging data was acquired by the scanner 14 by scanning an anatomical region of a patient. The set of medical imaging data was then stored in the data store 20 and passed from the data store 20 to the probability circuitry 24. In other embodiments, the medical imaging data may be received from any suitable data store or from the scanner 14 directly. The set of medical imaging data may comprise any suitable medical imaging data, for example any suitable two-, three- or four-dimensional medical imaging data.

In the embodiment of FIG. 2, the set of medical imaging data was obtained from a non-contrast CT (NCCT) scan of the brain of a patient. The brain of the patient was scanned because it was suspected that the patient had experienced a stroke. In other embodiments, the medical imaging data may have been obtained by scanning any anatomical region of a patient using any suitable imaging method. In further embodiments, the medical imaging data may have been obtained by optical imaging. For example, the medical imaging data may have been obtained by photography. The medical data may have been obtained by endoscopy. The medical imaging data may have been obtained by microscopy, for example for use in pathology. In other embodiments, any suitable medical data may be used.

At stage 32, the probability circuitry 24 applies a segmentation process to the medical imaging data. The applying of the segmentation process comprises applying a trained model to the medical imaging data. The trained model is a deep learning model that has been trained to perform a segmentation task. In the present embodiment, the deep learning model comprises a convolutional neural network. In other embodiments, any type of deep learning model may be used. In further embodiments, any suitable segmentation process may be used, which may or may not comprise deep learning. In further embodiments, any suitable process may be applied that produces a multiply-valued or continuously-valued output.

The segmentation process outputs a probability volume 34. The probability volume 34 may also be referred to as a heat map. The probability volume 34 comprises, for each of a set of locations in the medical imaging data, a probability that the tissue at that location belongs to a first class of tissue. For example, the locations may be voxel locations. In the present embodiment, the first class of tissue is tissue exhibiting signs of ischemia. Each probability value is a value between 0 and 1.

In some embodiments, the probability volume 34 may comprise multiple probabilities for each location, for example respective probabilities for each of a plurality of classes of tissue.

The probability circuitry 24 passes the probability volume 34 to the threshold circuitry 26.

At stage 38, the threshold circuitry 26 receives patient information. The patient information is associated with the patient who was scanned to obtain the set of medical imaging data. The patient information may also be described as patient-specific clinical information.

In the present embodiment, the patient information comprises a time since onset of stroke symptoms. In some circumstances, the time since onset may be approximated as a time since the patient was last known to be well.

In other embodiments, the patient information may comprise any patient information that may be relevant to thresholding of the probability volume.

The patient information may comprise or form part of the patient's medical records. For example, in some embodiments the patient information may comprise at least one of demographic information (for example, age and/or gender and/or ethnicity), vital sign information for example blood pressure information, or information about a patient's past or current medical conditions. The patient information may comprise information about lifestyle factors such as alcohol or smoking. The patient information may comprise information about a current or past medical condition or diagnosis.

The patient-specific information may comprise any information relating to the patient that is not part of the medical imaging data set, for example information other than the pixel and/or voxel intensity values that form the medical imaging data set. In some embodiments, the patient information may be found in metadata associated with the set of medical imaging data. For example, patient age and gender may be obtained from a DICOM data set.

The patient information may be obtained by any suitable method. In some embodiments, the patient information is input by a user. In some embodiments, the patient information is obtained using a machine learning model. The machine learning model is trained to extract patient information from a data set, for example to extract a desired item of patient information from the patient's medical records. An example of a model training process is described below with reference to FIG. 3.

At stage 40, the threshold circuitry 24 determines a threshold value based on the patient-specific information. The threshold value is a probability value between 0 and 1. In the present embodiment, the patient-specific information is the time since onset. The threshold circuitry 24 determines the threshold value using a threshold function which gives threshold value as a function of time of onset of stroke symptoms. The threshold function 42 is shown in FIG. 2.

The threshold circuitry 42 determines a patient-specific threshold value by inputting the patient-specific time since onset to the threshold function 42.

In the present embodiment, the threshold function is manually defined. The threshold function is defined using heuristics (clinical knowledge).

In some embodiments, the threshold function is defined by linear interpolation between a minimum and maximum value. Time since onset may be used to determine an interpolation amount. The threshold function may operate by interpolating between predefined values using patient-specific clinical information to determine interpolation rate. In alternative embodiments, any suitable interpolation function may be used, for example a higher order interpolation function.

In other embodiments, the threshold function is learned from data. The learning of a threshold function from data is described below with reference to FIG. 3.

In some embodiments, an approximate threshold value may be determined initially, and the threshold value may be obtained by modifying the approximate threshold value. For example, the threshold circuitry 26 may determine a value for an approximate threshold using receiver operating characteristic (ROC) analysis. In other embodiments, any suitable method may be used to obtain an approximate threshold. In some embodiments, a fixed, default threshold value may be used as the approximate threshold.

In some embodiments, a valid range may be set for the threshold value before the threshold value is determined. For example, a minimum and/or maximum value may be used to constrain the threshold value to ensure that it gives clinically valid results. We consider the clinical considerations that have been used to generate the threshold function 42 of this embodiment.

If an acute stroke is suspected, it is usual to obtain an NCCT scan of the brain of the patient. The NCCT scan is used to inform clinical decisions. In particular, the NCCT scan is used to inform the decision of whether tPA (tissue plasminogen activator) should be administered to the patient.

The decision to administer tPA must be considered carefully. In some circumstances, the administering of tPA may result in negative effects due to hemorrhage.

tPA is contraindicated in cases where the time since onset is large (for example, 4.5 hours). As time since onset increases, it may be expected that the negative effects of tPA start to outweigh the positive.

tPA is contraindicated in cases where the infarct is large (for example, an ASPECTS score above 7).

An ischemic infarct may show on an NCCT scan as a hypodense signal. As time since onset increases, it is typically the case that the hypodense signal becomes more easily visible.

It has been found that the sensitivity of detecting visible signs of ischemia on a CT modality is 40% to 60% within three hours after onset (Patel et al, Lack of clinical significance of early ischemic changes on computed tomography in acute stroke, JAMA. 2001 Dec. 12; 286(22):2830-8), increasing to 75% within six hours for some signs (Broderick, Recanalization therapies for acute ischemic stroke, Semin Neurol 1998; 18(4): 471-484).

In the present embodiment, a desired threshold value for the probability volume may be different in different clinical scenarios.

In the case of a large time since onset, a clinician wants to balance the risk of administering tPA with the possibility of hemorrhage. The clinician typically wants a confident prediction so that they can be sure of their decision. Furthermore, it is expected that a stronger hypodense signal is obtained over time. The ischemic infarct is likely to have a stronger hypodense signal. Therefore, at a large time since onset, a higher threshold value may be preferred. The higher threshold value may be considered to provide a higher confidence.

In the case of a small time since onset, a clinician may not be sure that ischemic stroke is occurring. Typically, the clinician will want some indication that the stroke will develop. The clinician will wish to exclude stroke mimics. The clinician is likely to want to view all possible ischemic regions so that they can made a decision themselves. In the case of a small time since onset, a large threshold may result in the clinician not seeing the infarct at all. Therefore, at a small time since onset, a lower threshold value may be preferred. The lower threshold value may be considered to provide a lower confidence.

Once the patient-dependent threshold value has been obtained, the threshold circuitry 26 applies the patient-dependent threshold value to the probability volume that was output by the probability circuitry 24. The threshold circuitry applies the patient-dependent threshold value to the probability volume to obtain a binary mask.

FIG. 2 shows three different binary masks 44, 46, 48 that are obtained by applying different threshold values to the same probability volume. The different threshold values are chosen to correspond to different times since onset.

Binary mask 44 corresponds to a time since onset of one hour. A low threshold value is chosen in accordance with the low time since onset. The low threshold value results in a large area being classified as ischemia.

Binary mask 46 corresponds to a time since onset of two hours. A higher threshold value is chosen, resulting in a smaller area being classified as ischemia.

Binary mask 48 corresponds to a time since onset of three hours. A still higher threshold value is chosen, resulting in a still smaller area being classified as ischemia.

In the embodiment of FIG. 2, the threshold circuitry 26 outputs a single threshold value which is used to obtain a single binary mask. The different masks 44, 46, 48 are shown as different examples corresponding to different threshold values in the method of FIG. 2. In other embodiments, the threshold circuitry 26 may output any suitable number of threshold values, which may be used to obtain any suitable number of binary masks.

At stage 50, an image is shown to the clinician using the modified threshold. The rendering circuitry 28 renders an image from the set of medical imaging data that was obtained at stage 30. The image is rendered using the binary mask corresponding to the threshold value output by the threshold circuitry 26. The rendered image is displayed to the clinician.

An area that is classified as ischemia is visibly distinguished in the rendered image. For example, an area of the brain that is classified as ischemia may be highlighted using a different color from the rest of the brain. The area classified as ischemia may be outlined. The area classified as ischemia may be labelled in the rendered image.

The image may comprise a heat map which is representative of probability values. A region that has been determined by thresholding may be presented along with the heat map. For example, the thresholded region may be overlaid on the heat map.

At a short time since onset, the clinician is shown a large area of tissue that might have signs of ischemia. The clinician can then review this large area for possible signs. At this time, the visible signs of ischemia are likely to be very subtle.

At a longer time since onset, the clinician is shown a smaller area of tissue, with higher confidence. At this time, signs of ischemia are likely to be more visible. It is important that the clinician has high confidence of ischemia if the clinician is considering using tPA.

At stage 52, the clinician is given control of the threshold value. The clinician may choose any threshold value that they prefer. The rendering circuitry 28 renders an image from the medical imaging data set using the threshold value that is selected by the clinician. A region that is classified as ischemia in accordance with the new threshold value is visually distinguished in the rendered image.

The clinician may choose a different threshold to that obtained by the threshold function. The clinician may view all of the information if they want. For example, the display may show precomputed sensitivity, specificity and/or values for other parameters.

In many cases, the threshold value may only be determined automatically. The clinician may not need to adjust the threshold value. Stage 52 may be considered to be optional.

In the above description, we have referred generally to a clinician. However, the method as described in FIG. 2 may be capable of being used by any user, for example any doctor, radiographer, or researcher.

By using patient information to choose the threshold, a different threshold may be applied to each patient. In the case of acute stroke ischemia detection, the time since onset is used to adjust the threshold to match the clinical decision that has to be made (in this case, the decision of whether to administer tPA).

In some circumstances, it may be difficult to include patient information as an input to a machine learning algorithm, for example a segmentation algorithm. In the embodiment of FIG. 2, the thresholding step is used as a secondary stage through which patient information can be introduced in combination with the initial segmentation. A result may therefore be obtained which is dependent on the patient-specific information.

The method of FIG. 2 may be particularly useful in ischemia, because signs of ischemia may be very subtle at an early stage. In other embodiments, a method similar to that of FIG. 2 may be applied to signs of other diseases or medical conditions. For example, the method of FIG. 2 may be useful in any segmentation task in which patient-specific clinical information may affect an algorithm's certainty of a result and/or a size of the segmentation. The method of FIG. 2 may be particularly useful where boundaries of a target to be segmented may not be clear.

In the embodiment of FIG. 2, a threshold function is used to obtain a threshold value based on time of onset. In other embodiments, a threshold function may determine threshold value based on any suitable patient-specific information. A threshold function may determine threshold value based on multiple items of patient-specific information, for example time since onset plus age plus gender.

In some embodiments, the method of FIG. 2 is applied to a segmentation of liver fibrosis. The patient-specific information used to determine a threshold may comprise, for example, information regarding whether the patient has a history of alcohol abuse, or details of a patient's history of alcohol abuse (for example, a duration of alcohol abuse). Information regarding alcohol abuse may be used to segment a probability volume for the segmentation of liver fibrosis.

In some embodiments, the method of FIG. 2 is applied to a segmentation of arterial calcium or plaque. The patient-specific information used to determine a threshold may comprise information on factors relevant to the presence of arterial calcium or plaque, for example a patient's history of smoking and/or a patient's weight.

In some embodiments, the method of FIG. 2 is applied to a segmentation of visceral fat. The patient-specific information used to determine a threshold may comprise, for example, information regarding the patient's ethnicity.

In some embodiments, the method of FIG. 2 is applied to segmentation of the prostate. The patient-specific information used to determine a threshold may comprise, for example, information regarding the patient's age.

In some embodiment, the method of FIG. 2 is applied to segmentation of a tumor, for example a cancer tumor. The patient-specific information used to determine a threshold may comprise, for example, information on the type of cancer and/or information on treatments that have been given to the patient.

In some circumstances, tumors can exhibit a pseudoresponse or pseudoprogression in which features seen in imaging do not match the true change in the tumor. For example, a pseudoresponse or pseudoprogression may be due to a recent treatment. Knowledge of the recent treatment may be used to adjust the threshold, for example to account for the possibility of pseudoresponse or pseudoprogression. Different threshold values or changing threshold values may be used to demonstrate an uncertainty in an algorithm.

In the embodiment of FIG. 2, a single image is displayed to the user. The threshold function is used to obtain a single threshold value based on time of onset. The single image is rendered using a binary mask obtained using the single threshold value.

In other embodiments, multiple threshold values are determined from a single medical imaging data set. For example, different threshold values may be applicable for different clinical uses or applications. In some embodiment, the threshold circuitry 26 obtains a range of threshold values around a preferred threshold value. In some embodiments, a range of predetermined threshold values are used.

Regions corresponding to each of the multiple threshold values may be displayed to the user. Multiple different regions may be visually indicated in a single image, for example by using different colors or patterns. Multiple regions may be shown as multiple layers. Multiple regions may be shown like a topographic map having contours.

In some embodiments, the threshold circuitry 26 obtains threshold values corresponding to different confidence levels. The rendered image visually indicates confidence intervals. Confidence intervals may be shown using a visual indication such as, for example, color or distance between contours.

In other embodiments, multiple images may be rendered, with each of the images showing a region corresponding to a different one of the threshold values.

In some embodiments, the user is presented with a screen showing the multiple images. The user may also be shown the associated threshold value for each of the images. The user may select which of the images they wish to examine. For example, the user may click on one of the images to enlarge it. By clicking on the image, the user may be considered to select one of the multiple threshold values.

In some embodiments, each image is provided with a slider for the or each threshold value used to render the image. Adjusting the slider position will show the effect of adjusting the threshold value.

In some embodiments, a display may use multiple threshold values to provide a visual indication of a measure of uncertainty in an algorithm. Multiple threshold values may be used to show a range of possible results. Multiple threshold values may be used to show results under different assumptions, for example results with or without a suspected pseudoresponse or pseudoprogression.

In some embodiments, threshold values are determined for a maximum and minimum of a clinically valid range of a patient information parameter. The rendered image or images show the segmented regions for the maximum and minimum values. The rendered image or images may also show a predicted region for the actual patient-specific value for the parameter and/or a region for a threshold value selected by a user. The maximum and minimum regions may be visually indicated, for example by dotted contours.

Each segmented region in a rendered image may have an associated indication (for example a label or key) showing the threshold value used to obtain that region. Each segmented region may have an associated indication showing the patient-specific information (for example, time of onset) used to obtain that segmented region. Each segmented region may have an associated indication showing an estimate value for at least one statistical measure for the region, for example an estimated sensitivity and/or specificity.

In some embodiments, multiple pieces of clinical data are considered in the calculation of the threshold value. The calculation may be designed such that individual pieces of data can be ignored. Multiple threshold values may be calculated, each corresponding to a different combination of clinical information. For example, a calculation of threshold value may take into account age, gender and smoking history. A first threshold value may be determined based only on the patient's age, without taking into account gender or smoking history. A second threshold value may be determined based only on gender without taking into account age or smoking history. A third threshold value may be determine based only on smoking history without taking into account age or gender.

Further threshold values may be determined based on different combinations of age, gender and/or smoking history. This effectively results in multiple segmentations, one for each possible combination of data.

Of these options the most important may be the segmentation considering all patient-specific data and the segmentation considering none of the patient-specific data.

A user may be given the ability to exclude different pieces of clinical information from consideration. For example, the user may be provided with a checkbox corresponding to each piece of data. By checking and unchecking the checkbox, the user may indicate which pieces of information they want to be considered in determining the threshold, and therefore the segmented region. A user may therefore easily explore the space of different clinical information, and how the segmentation changes when the clinical information is or is not taken into account.

In some embodiments, a visual indication on the display may tell the user that the medical application uses a threshold value that is adapted based on patient-specific information. The visual indication may tell the user which patient-specific information is being used for thresholding. For example, the visual indication may tell the user that a threshold value has been selected based on time of onset. The display may include a warning indication that explains that the displayed result is highly dependent on changes in the threshold value.

In the embodiment of FIG. 2, the threshold function is manually defined. In other embodiments, the threshold function is learned from data. FIG. 3 is a flow chart showing in overview a method of training a threshold function.

At stage 60, the training circuitry 29 receives training data. The training data has been gathered through real-time usage of a medical application having a segmentation process, in which the clinician can select a threshold value with which to view the results of the segmentation process. It may be assumed that clinicians most often select the threshold values that they consider to be more useful. The training circuitry 29 or other circuitry may track the thresholds that the clinicians find most useful when making decisions, along with the patient-specific information for the associated cases.

The training data comprises a plurality of data samples. Each data sample comprises patient-specific information relating to a set of medical imaging data. For example, the patient-specific information may comprise time of onset. Each data sample also comprises at least one threshold value that was selected by a clinician for use when viewing the set of medical imaging data. In some embodiments, the data sample also comprises the set of medical imaging data. In further embodiments, the data sample comprises data about a patient outcome. For example, the data sample may comprise information about whether tPA was administered to the patient. The data sample may comprise information about whether the patient responded well or poorly to an administration of tPA.

The threshold values that were selected by clinicians provide a ground truth for use in training.

At stage 62, the training circuitry 60 trains a threshold function in dependence on the training data. Any suitable method of training the threshold function may be used. In the embodiment of FIG. 3, the threshold function is trained to output a threshold value in dependence on time of onset. In further embodiments, the threshold function may be trained to output a threshold value in dependence on additional or alternative patient-specific information. In further embodiments, the threshold function may be trained to output a threshold value in dependence on an outcome variable that is representative of a clinical outcome.

At stage 64, the threshold circuitry 26 uses the trained threshold function to threshold a new probability volume which does not form part of the training data. The threshold circuitry 29 inputs patient-specific information into the trained threshold function. The trained threshold function outputs at least one threshold value.

In one simple embodiment of stage 62, the training circuitry 29 plots selected threshold values against values for one or more items of patient-specific information. The training circuitry 29 fits a curve to the resulting plot.

In a more complex embodiment, the training function comprises machine learning model, for example a convolutional neural network. The training circuitry 29 provides the training data as an input to the machine learning model. In this case, the output of stage 62 is a trained model. At stage 64, the threshold circuitry 26 provides the patient-specific information to the trained model. The trained model outputs at least one threshold value.

In some embodiments, the training data does not include ground truth data. The training circuitry 29 trains the threshold function using an unsupervised learning process. In other embodiments, the training data includes some labelling and/or annotation, but does not include ground truth threshold values. Weak supervision may be used in training the threshold function.

In embodiments described above, a segmentation process is applied to medical imaging data that is obtained by scanning with a scanner.

In other embodiments, the medical imaging data may comprise any suitable type of medical imaging data. For example, the medical imaging data may be optical data, for example from photography. The medical imaging data may be pathology data. The medical data may be obtained from a procedure in which an imaging device is inserted into the patient's body, for example laparoscopy.

In further embodiments, a method as described above with reference to FIG. 2 may be used to perform any suitable process which produces multiply valued or continuously-valued output, for example any suitable machine learning process. The process may not comprise a segmentation. The process may comprise processing any suitable data items of a data set to obtain any multiply-valued or continuously-valued output. The output may not be a probability. Any suitable threshold value may be applied to the output.

The method of FIG. 2 may be performed using medical data relating any human or animal subject. The method of FIG. 2 may be used in the detection and/or diagnosis of any suitable disease or medical condition. For example, the method of FIG. 2 may be used to view damage to the heart that has been caused by a heart attack.

In further embodiments, the method of FIG. 2 may be used to perform a thresholding of the output of any suitable process having a multiply-valued or continuously-valued output. The process may comprise processing any data items in a data set. The thresholding may be based on any suitable inputs that do not form part of the data items.

Certain embodiments may provide a method of processing medical imaging data comprising: applying a segmentation process to the data to obtained continuously-valued segmentation outputs; determining at least one threshold dependent on patient-specific clinical information; and applying the determined at least one threshold to the continuously-valued segmentation outputs to obtain thresholded segmentation outputs.

Certain embodiments provided a medical imaging apparatus comprising a segmentation algorithm with continuous outputs; a specific range (threshold) that is used to group the algorithm outputs; and a threshold calculation method which is dependent on patient-specific clinical information.

The segmentation algorithm may be a convolutional neural network. The threshold calculation may be determined by training a machine learning algorithm. The machine learning algorithm may be trained to maximize an outcome variable which is some way dependent on the algorithm output. Multiple thresholds may be used to group outputs into more than one group.

The results may be viewed within a medical imaging application. The original numerical results may also be viewed. The threshold may be manually adjusted. The grouped results may contain an indicator of continuous value (for example, a heat map).

The threshold function may operate by interpolating between predefined values using patient-specific clinical information to determine interpolation rate.

The algorithm may be predicting stroke ischemia. The patient-specific clinical information may include time since onset of stroke.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A data processing apparatus comprising processing circuitry configured to:
apply a process to a medical data set to obtain multiply-valued and/or continuously-valued process outputs;
determine at least one threshold value dependent on patient-specific clinical information; and
apply the determined at least one threshold value to the process outputs to obtain thresholded process outputs.

2. An apparatus according to claim 1, wherein the medical data set comprises medical imaging data, the process comprises a segmentation process, and the process outputs are segmentation outputs.

3. An apparatus according to claim 2, wherein the segmentation process comprises applying a segmentation algorithm for determining the presence and/or location of at least one anatomical feature, pathology or other feature of interest; the data comprises a set of data points each corresponding to a respective location; and the multiply-valued and/or continuously-valued segmentation outputs comprise, or can be used to determine, a probability for each location or group of locations whether the anatomical feature or other feature of interest is present at that location or group of locations.

4. An apparatus according to claim 3, wherein the thresholded segmentation outputs comprise, for each location or group of locations an indication of whether or not said at least one anatomical feature, pathology or other feature of interest is present or absent according to said determined threshold value.

5. An apparatus according to claim 3, wherein the pathology comprises stroke ischemia.

6. An apparatus according to claim 1, wherein the patient-specific clinical information comprises time since onset of stroke.

7. An apparatus according to claim 1, wherein the patient-specific clinical information comprises at least one of demographic information, age, gender, ethnicity, height, weight, blood pressure information, vital signs information, information about a medical condition of the patient, information about a diagnosis, information about a medical treatment, information about a lifestyle factor of the patient, information about alcohol use, information about smoking.

8. An apparatus according to claim 1, wherein the process uses or comprises a convolutional neural network.

9. An apparatus according to claim 1, wherein the determining of the threshold is performed using a process obtained by training a machine learning algorithm.

10. An apparatus according to claim 9, wherein the machine learning algorithm is trained to maximize an outcome variable which is at least partially dependent on outputs of the process.

11. An apparatus according to claim 1, wherein the processing circuitry is further configured to obtain the patient-specific clinical information using a trained model.

12. An apparatus according to claim 1, wherein the at least one threshold comprises a plurality of thresholds that are used to group outputs into more than one group.

13. An apparatus according to claim 1, wherein the processing circuitry is further configured to display a visual representation of at least some of the thresholded process outputs and/or at least some of the multiply-valued and/or continuously-valued process outputs.

14. An apparatus according to claim 13, wherein the visual representation further comprises a visual representation of at least some of the multiply-valued and/or continuously-valued process outputs overlaid on or otherwise combined with the visual representation of at least some of the thresholded process outputs.

15. An apparatus according to claim 1, wherein the at least one threshold value comprises multiple different threshold values, and wherein the processing circuitry is further configured to display a visual representation of a plurality of regions, each region corresponding to thresholded process outputs obtained using a respective one of the multiple threshold values.

16. An apparatus according to claim 1, wherein the processing circuitry is further configured to receive at least one user input and to adjust the determined threshold or thresholds based on the at least one user input.

17. An apparatus according to claim 16, wherein the at least one user input comprises at least one of: a selection of a threshold value, a movement of a slider that is representative of threshold value, a selection of at least one of a plurality of images, a selection of at least one piece of patient-specific clinical information.

18. A method for processing medical data comprising:
applying a process to a medical data set to obtain multiply-valued and/or continuously-valued process outputs;
determining at least one threshold value dependent on patient-specific clinical information; and
applying the determined at least one threshold value to the multiply-valued and/or continuously-valued classification outputs to obtain thresholded classification outputs.

19. A training apparatus comprising processing circuitry configured to:
obtain training data comprising patient-specific clinical information; and
train a machine learning algorithm to predict threshold values for continuously-valued outputs of a process by performing a training process using the training data, the training process comprising training the machine learning algorithm to predict threshold values based on patient-specific clinical information.

20. An apparatus according to claim 19, wherein the training data further comprises ground truth threshold values and/or wherein the machine learning algorithm is trained to maximize an outcome variable which is at least partially dependent on outputs of the process.
